(19) United States
(12) Patent Application Publication
(10) Pub. No.: US 2023/0131650 A1
(43) Pub. Date: Apr. 27, 2023

(54) IMMUNOGENIC CONJUGATE INTENDED TO INDUCE AN IMMUNE RESPONSE DIRECTED AGAINST INTERLEUKIN-6

(71) Applicant: PEPTINOV, PARIS (FR)

(72) Inventors: Lucille DESALLAIS, DEUIL LA BARRE (FR); Jean-Pierre SALLES, EGUILLES (FR); Jean-Francois ZAGURY, LE VESINET (FR)

(21) Appl. No.: 17/758,279

(22) PCT Filed: Dec. 31, 2020
(86) PCT No.: PCT/EP2020/088090

§ 371 Date: Jan. 6, 2023

(30) Foreign Application Priority Data
Dec. 31, 2019 (EP) 19306799.8

(51) Int. Cl. A61K 39/00; C07K 14/54; C07K 14/715; C07K 14/34; A61P 37/02

(52) U.S. Cl. CPC A61K 39/00114; C07K 14/5412; C07K 14/7155; C07K 14/34; A61K 39/001119; A61K 39/0008; A61P 37/02; C07K 2319/55; A61K 2039/6037; A61K 2039/627

(57) ABSTRACT

The present invention relates to an immunogenic conjugate comprising:
a carrier protein and
at least one polypeptide having at most 100 amino acids comprising a sequence of 5 to 50 amino acids of interleukin 6 (IL-6) or IL-6 receptor (IL-6R), or a variant sequence having at least 75% identity with the sequence of 5 to 50 amino acids of IL-6 or IL-6R, wherein the polypeptide is covalently linked to the carrier protein and the carrier protein is a non-toxic mutant diphtheria toxin.

### FIELD OF THE INVENTION

The present invention relates to an immunogenic conjugate, a pharmaceutical composition, in particular a vaccine composition, comprising it, and its use in a method of preventing or treating diseases related to overexpression or overproduction of interleukin-6.

### TECHNICAL BACKGROUND

Interleukin-6 (IL-6) is an essential cytokine in particular involved in the regulation of immune cell proliferation and differentiation.

IL-6 is strongly overproduced during inflammatory processes, and this overproduction is observed in many diseases such as infections, acute or chronic inflammatory diseases, and also cancer.

IL-6 thus appears to be the driving signal in several inflammatory diseases. Preclinical studies in animal models of human diseases have shown that blocking IL-6 activity alleviates symptoms or even completely prevents the onset of the disease.

The IL-6 signalling cascade offers several alternatives for therapeutic intervention, ranging from biologics that block the cytokine or its receptor outside the cell to small chemical molecules that target the kinases and transcription factors involved inside the cell. Interestingly, in patients, antibodies that target IL-6 can be used in significantly lower amounts compared to antibodies that block the IL-6 receptor (IL-6R). This is because the soluble IL-6 receptor (sIL-6R) is present in serum at high concentrations, and because these soluble receptors also take up neutralizing anti-IL-6R antibodies, a lot of the latter are needed so that they can also block IL-6R membrane receptors on the cell surface and thus prevent IL-6 signalling.

IL-6 concentrations are relatively low in healthy individuals (a few picograms per milliliter of serum) and antibodies directly targeting the IL-6 cytokine need only capture newly synthesized and released IL-6 molecules to be active. Direct blockade of IL-6 does not interfere with other cytokines that can signal via the IL-6R and therefore provides a very specific inhibition of IL-6.

In recent years, several classes of therapeutics targeting the components of the IL-6 signalling pathway have been developed. The efficacy of therapeutic interventions neutralizing IL-6 or interfering with its signalling has demonstrated the major deleterious role of IL-6 in a number of diseases.

For example, IL-6R blocker tocilizumab, the first monoclonal antibody developed against the IL-6 pathway, is now approved for the treatment of moderate to severe active rheumatoid arthritis (RA) in adults. Other monoclonal antibodies targeting IL-6R are in development: sarilumab, approved in rheumatoid arthritis; NI-1201, currently in preclinical trials; and vobarilizumab, currently in phase II clinical trials for the treatment of rheumatoid arthritis and systemic lupus erythematosus.

Several monoclonal antibodies directly targeting IL-6 have also been developed, including: sirukumab, for the treatment of rheumatoid arthritis, depression and lupus nephritis (phase II); olokizumab, for the treatment of rheumatoid arthritis and Crohn's disease; clazakizumab, for the treatment of organ transplant rejection; and siltuximab, which is indicated for the treatment of Castleman's disease and is also intended to be used in the treatment of multiple myeloma.

However, all these passive immunotherapy treatments, i.e. involving the administration of antibodies, particularly monoclonal antibodies, require fairly frequent and long-term administration, which entails major constraints in terms of both patient comfort and costs.

In order to overcome these drawbacks, an approach to the prevention and treatment of IL-6 related diseases based on active (or vaccine) immunotherapy has been developed. This approach relies on the induction of a humoral immune response using immunogenic polypeptides derived from IL-6 leading to the production of anti-IL-6 antibodies by the patient himself.

Desallais et al. (2016) Sci. Rep. 6:19549 thus describe the production of neutralizing human anti-IL-6 antibodies in cynomolgus monkeys that were administered an IL-6-derived polypeptide. However, the levels of antibodies obtained remained moderate. An objective of the present invention is therefore to improve the production of anti-IL-6 antibodies in active immunotherapy.

### SUMMARY OF THE INVENTION

The present invention arises from the unexpected finding, by the inventors, that conjugation of IL-6 derived polypeptides to the carrier protein CRM-197 increases the production of anti-IL-6 antibodies in the individual to whom the conjugate is administered compared to other carrier proteins.

Thus, the present invention relates to an immunogenic conjugate comprising:
a carrier protein and
at least one polypeptide having at most 100 amino acids comprising a sequence of 5 to 50 amino acids of interleukin 6 (IL-6) or interleukin 6 receptor (IL-6R), or a variant sequence having at least 75% identity with the sequence of 5 to 50 amino acids of IL-6 or IL-6R,

wherein the at least one polypeptide is covalently linked to the carrier protein and the carrier protein is a non-toxic mutant diphtheria toxin.

In a particular embodiment of the immunogenic conjugate as defined above, the polypeptide is bound to the carrier protein via a non-peptide coupling agent.

The present invention also relates to the immunogenic conjugate as defined above, for use in a method of therapeutic treatment, for use in a method of preventing or treating a disease related to overproduction or overexpression of IL-6, or for use in a method of vaccination against IL-6 or IL-6R, or in a method of inducing an immune response against IL-6 or IL-6R, in an individual.

The present invention also relates to a method of preventing or treating a disease related to overexpression or overproduction of IL-6 in an individual, wherein the individual is administered a prophylactically or therapeutically effective amount of an immunogenic conjugate as defined above.

The present invention also relates to a method of vaccinating against IL-6 or IL-6R or inducing an immune response against IL-6 or IL-6R in an individual, wherein the individual is administered an effective amount of an immunogenic conjugate as defined above.

The present invention also relates to a pharmaceutical composition, in particular a vaccine composition, comprising as active substance at least one immunogenic conjugate as defined above, optionally in association with at least one pharmaceutically acceptable vehicle and/or excipient.

In a particular embodiment of the invention, the pharmaceutical composition, in particular vaccine, as defined above further comprises at least one adjuvant.

The present invention also relates to the pharmaceutical composition, in particular vaccine, as defined above, for use in a method of therapeutic treatment, for use in a method of preventing or treating a disease related to overexpression or overproduction of IL-6, or for use in a method of vaccination against IL-6 or IL-6R, or in a method of inducing an immune response against IL-6 or IL-6R, in an individual.

The present invention also relates to a method of preventing or treating an IL-6 overexpression related disease in an individual, wherein the individual is administered a prophylactically or therapeutically effective amount of a pharmaceutical composition as defined above.

The present invention also relates to a method of vaccinating against IL-6 or IL-6R or inducing an immune response against IL-6 or IL-6R in an individual, wherein the individual is administered an effective amount of a pharmaceutical composition as defined above.

The present invention also relates to a process for the preparation of an immunogenic conjugate as defined above, comprising a step of covalently linking at least one polypeptide having at most 100 amino acids comprising a 5 to 50 amino acid sequence of interleukin 6 (IL-6) or IL-6 receptor (IL-6R) or a variant sequence having at least 75% identity to the 5 to 50 amino acid sequence of IL-6 or IL-6R, with a carrier protein that is a non-toxic mutant diphtheria toxin.

### DESCRIPTION OF THE FIGURE

FIG. 1 represents the mean titer₅₀ of anti-hIL-6 antibody (y-axis, arbitrary units) as a function of time (x-axis, days) of 6 groups of 5 rabbits respectively immunized with doses of 5 μg (group 1, small circles), 7 μg (group 2, squares), 10 μg (group 3, upward-oriented triangles), 15 μg (group 4, downward-oriented triangles), 50 μg (group 5, diamonds) of polypeptide, or by CRM197 carrier protein (group 6, large circles), at days 0, 13, 43 and 73 (arrows).

### DETAILED DESCRIPTION

As a preliminary remark, it should be noted that the term “consisting of” means “constituted by”, i.e. when an object “consists of” an element or several elements, the object cannot include other elements than those mentioned. In contrast, the term “comprising” means “including”, “containing” or “encompassing”, i.e. when an object “comprises” an element or elements, other elements than those mentioned can also be included in the object. In other words, when an object “comprises” an element or elements, it consists of the element(s) and possibly of other elements than these.

### Polypeptide

### Definitions

Interleukin 6 (IL-6), also sometimes referred to as B-cell stimulatory factor 2 (BSF-2), cytotoxic T-cell differentiation factor (CDF), hybridoma growth factor, or interferon 0-2 (IFN-β-2), is well known to the skilled person. Numerous sequences of IL-6 from various animal species are available in sequence databases. As an example, a human IL-6 is described in the UniProt/Swissprot database as P05231 (SEQ ID NO: 1). As an example also, the alpha subunit of the human IL-6 receptor is described in the UniProt/Swissprot database under the reference P08887 (SEQ ID NO: 2).

As used herein, the terms “peptide” and “polypeptide” are used in their broadest sense to refer to a molecule of two or more amino acid residues. The amino acid residues may be linked by peptide bonds, or alternatively by other bonds, e.g. ester, ether, etc. However, the amino acid residues are preferably linked together by peptide bonds.

As used herein, the terms “amino acid” and “amino acid residue” encompass natural and non-natural or synthetic amino acids, including D- and L-forms, and amino acid analogues. An “amino acid analogue” is to be understood as a non-naturally occurring amino acid that differs from a corresponding naturally occurring amino acid in one or more atoms. For example, an amino acid analogue of cysteine may be homocysteine. However, the amino acids or amino acid residues are preferably naturally occurring.

The polypeptide according to the invention is such that it shall be able to elicit an immune response directed against IL-6 or IL-6R; that is to say, administration of such a polypeptide bound to a carrier protein, such as CRM-197 or KLH for example, to an animal, such as a mouse, rat or rabbit for example, causes the production of antibodies directed against IL-6 or IL-6R. The polypeptide according to the invention is therefore immunogenic and comprises one or more epitopes of IL-6 or IL-6R. It is well known to the skilled person how to determine whether an antibody is directed against IL-6 or IL-6R, in particular by implementing an ELISA test.

As understood here, the percentage of identity between two peptide sequences can be determined by performing an optimal alignment along the entire length of the sequences, determining the number of aligned positions for which the amino acids are identical in each sequence and dividing this number by the total number of amino acids in the longer of the two sequences. The optimal alignment is the one that gives the highest percentage of identity between the two sequences.

As used here, the terms “overproduction” and “overexpression” of IL-6 are considered equivalent and mean that IL-6 is present in the body of an individual to be treated in a concentration beyond normal or at a non-physiological or pathological concentration.

Length

Preferably, the polypeptide according to the invention comprises at most 100, 90, 80, 70, 60, 50, 40, 30, 25, 24, 23, 22, 21 or 20 amino acids. Preferably, the polypeptide according to the invention comprises at least 5, 6, 7, 8, 9, 10, 12, 15, 18, 20, 40, 50, 60, 70, 80 or 90 amino acids. Preferably, the polypeptide according to the invention comprises from 10 to 40 amino acids, more preferably from 15 to 35 amino acids.

IL-6

Preferably, the IL-6 according to the invention is selected from the group consisting of human IL-6 (hIL-6), mouse IL-6, monkey IL-6, in particular macaque IL-6, horse IL-6, dog IL-6, cat IL-6, or rabbit IL-6. Particularly preferably the IL-6 according to the invention is human IL-6.

Sequence

Preferably, the polypeptide according to the invention comprises, or consists of, a sequence of at least 5, 6, 7, 8, 9, 10, 11 or 12 amino acids of IL-6 or IL-6R. Preferably, the polypeptide according to the invention comprises, or consists of, a sequence of at most 50, 45, 40, 35, 30, 25, 20, 19, 18, 17, 16, 15, 14, 13, 12, 11 or 10 amino acids of IL-6 or IL-6R. Preferably, the polypeptide according to the invention comprises, or consists of, a sequence of 6 to 40, 7 to 35, or 8 to 30 amino acids of IL-6 or IL-6R.

Preferably, the polypeptide according to the invention comprises, or consists of, a sequence of 7 to 35 amino acids of IL-6 or IL-6R, or a variant sequence having at least 90% identity with the sequence of 7 to 35 amino acids of IL-6 or IL-6R.

In particular, the polypeptide according to the invention may be as described in International application WO2013/021284, which is incorporated herein by reference. Preferably, the amino acid sequence of IL-6 or IL-6R according to the invention comprises at least 5, 6, 7, 8, 9, 10, 11, or 12 amino acids, or all, of sequences 58-78, 73-94, 96-111, 122-141, or 172-189 of IL-6. Preferably, the amino acid sequence of IL-6 or IL-6R according to the invention consists of 5, 6, 7, 8, 9, 10, 11, or 12 amino acids, or all, of sequences 58-78, 73-94, 96-111, 122-141, or 172-189 of IL-6.

Preferably, the amino acid sequence of IL-6 or IL-6 according to the invention comprises at most 21, 20, 19, 18, 17, 16, 15, 14, or 13 amino acids of sequences 58-78, 73-94, 96-111, 122-141, or 172-189 of IL-6.

The above numbering is based on the reference sequence containing the signal peptide, and presented as an example for human IL-6 and murine IL-6:

| | Human IL-6 (hIL-6): |
|---|---|
| | Sequence 58 to 78: |
| (SEQ ID NO: 3) | |
| | RYIIDGISALRKETCNKSNMC |
| | Sequence 73 to 94: |
| (SEQ ID NO: 4) | |
| | NKSNMCESSKEALAENNLNLPK |
| | Sequence 96 to 111: |
| (SEQ ID NO: 5) | |
| | AEKDGCFQSGFNEETC |
| | Sequence 122 to 141: |
| (SEQ ID NO: 6) | |
| | FEVYLEYLQNRFESSEEQAR |
| | Sequence 172 to 189: |
| (SEQ ID NO: 7) | |
| | NASLLTKLQAQNQWLQDM |
| | Sequence 196 to 212: |
| (SEQ ID NO: 8) | |
| | RSFKEFLQSSLRALRGM |
| | Murine IL-6 (mIL-6): |
| | Sequence 58 to 78: |
| (SEQ ID NO: 9) | |
| | VLWEIVEMRKELCNGNSDCMN |
| | Sequence 73 to 94: |
| (SEQ ID NO: 10) | |
| | NSDCMNNDDALAENNLKLPEIG |
| | Sequence 96 to 111: |
| (SEQ ID NO: 11) | |
| | NDGCYQTGYNGEICLL |
| | Sequence 122 to 141: |
| (SEQ ID NO: 12) | |
| | SYLEYMKNNLKDNKKDKARV |
| | Sequence 172 to 189: |
| (SEQ ID NO: 13) | |
| | ALLTDKLESQKEWLRTKT |
| | Sequence 196 to 211: |
| (SEQ ID NO: 14) | |
| | SLEEFLKVTLRSTRQT. |

Thus, preferably, the polypeptide defined above comprises at least 5, 6, 7, 8, 9, 10, 11 or 12 amino acids, or all, or consists of 5, 6, 7, 8, 9, 10, 11 or 12 amino acids, or all, of an IL-6 sequence selected from the group consisting of the sequences:

RYIIDGISALRKETCNKSNMC,
NKSNMCESSKEALAENNLNPK,
AEKDGCFQSGFNEETC,
FEVYLEYLQNRFESSEEQAR,
NASLLTKLQAQNQWLQDM,
and
SFKEFLQSSLRALRQM.

Furthermore, the polypeptide according to the invention may comprise several repeats, for example 2, 3, 4, 5, 10 or 20 repeats, respectively of the IL-6 or IL-6R sequence or the variant sequence defined above.

Variant Sequence

A variant sequence according to the invention, which has at least 75% identity with the IL-6 or IL-6R sequence according to the invention, will preferably have at least 80%, 85%, 90%, 95%, or 98% identity with the IL-6 or IL-6R sequence according to the invention. Very preferably, this variant sequence will have at most 50, 45, 40, 35, 30, 25, 20, 19, 18, 17, 16, 15, 14, 13, 12, 11 or 10 amino acids.

Cyclization

Preferably, the polypeptide according to the invention is cyclized.

The polypeptide according to the invention can be cyclized, i.e. the entire polypeptide forms a ring or a portion thereof forms a ring, according to methods of any kind known to the skilled person.

Depending on the functional groups present in the polypeptide, this cyclization can take place in several different ways, such as: from its C-terminal end to its N-terminal end, from its N-terminal end to a side chain, from a side chain to its C-terminal end, or between two side chains. The side chain moieties involved in cyclization notably include —NH₂, —COOH and —SH groups.

Among the various modalities of cyclization of polypeptides, can be mentioned the formation of a peptide bond between the two N- and C-terminal ends of the polypeptide, lactamization, lactonization or the formation of a disulfide bond between two cysteines (C) of the polypeptide. In particular, when an inter-cysteine disulfide bond is formed, i.e. between the —SH radicals of two cysteines, the cysteines may already be present in the IL-6 or IL-6R sequence, or they may be added within these sequences, to form variant sequences, or at their N- and/or C-terminus(es).

Modifications

The polypeptide according to the invention may comprise post-translational modifications, such as glycosylations, methylations, acylations, in particular by fatty acids or by an acetyl group, amidations, or phosphorylations. For example, the N-terminus of the polypeptide according to the invention may be acetylated or its C-terminus may be modified by amidation.

Additional Sequences

Preferably, the polypeptide according to the invention comprises one or more additional sequences, in addition to the IL-6 or IL-6R sequence according to the invention.

These additional sequences according to the invention may in particular provide physicochemical characteristics allowing an improved structural presentation or an improved solubility of the polypeptide according to the invention compared to a similar polypeptide but which would not comprise these additional sequences.

The additional sequences according to the invention may in particular comprise one or more peptide linker sequences, useful for binding in particular to a carrier molecule. Such peptide linker sequences typically comprise from 1 to 10, in particular from 1 to 6, and in particular 2 to 5 amino acids.

A particularly preferred linker sequence according to the invention is the sequence EGEZ (SEQ ID NO: 15), wherein Z is an amino acid allowing binding to the carrier protein, in particular selected from the group consisting of cysteine (C), tyrosine (Y), and lysine (K), more particularly from the group consisting of cysteine (C) and tyrosine (Y). Particularly preferably Z is tyrosine (Y). Advantageously, the EGEZ sequence improves solubility while maintaining good immune responses as shown in Examples 4, 5, and 6 below.

Another preferred linker sequence is an amino acid Z allowing binding to the carrier protein, in particular selected from the group consisting of cysteine (C), tyrosine (Y), and lysine (K), more particularly from the group consisting of cysteine (C) and tyrosine (Y). Particularly preferably Z is tyrosine (Y).

Alternatively, the additional sequence(s) may also include epitopes belonging to proteins other than IL-6 or IL-6R, allowing to elicit or generate an immune response directed against these other proteins.

Furthermore, the additional sequences according to the invention may also comprise exogenous, preferably universal, T epitope(s) sequences. Advantageously, these additional T epitope sequences allow enhancing the immunogenicity of the polypeptide according to the invention.

Preparation

The polypeptide according to the invention can be prepared by any method known in the state of the art and in particular by chemical synthesis. It is also possible to prepare it by the recombinant route in eukaryotic or prokaryotic cells.

### Carrier Protein

The carrier protein according to the invention is a non-toxic mutant diphtheria toxin, i.e. a diphtheria toxoid obtained by mutagenesis. Advantageously, this mutant diphtheria toxin is pharmaceutically acceptable.

The carrier protein according to the invention is preferably selected from the group consisting of CRM197, CRM176, CRM228, CRM45, CRM9, CRM102, CRM103, and CRM107.

Particularly preferably, the carrier protein according to the invention is CRM197. CRM197 is a genetically detoxified form of diphtheria toxin. It has a unique mutation at position 52, substituting a glycine (G) with a glutamic acid (E), which causes the loss of ADP-ribosyltransferase activity. However, it retains all the amine radicals of lysines (K) available for conjugation. CRM197 has 535 amino acids (58.4 kDa) and consists of two subunits linked by disulfide bridges. It is described under the GenBank accession number 1007216A (SEQ ID NO: 16). It can be produced recombinantly in Corynebacterium diphtheria, and also by other bacteria such as Pseudomonas fluorescens or Escherichia coli.

### Coupling Agent

Preferably the coupling agent according to the invention is non-peptidic.

The coupling agent can be heterobifunctional, such as N-γ-maleimidobutyryl-oxysuccinimide (GMBS) ester and the sulfo-GMBS derivative, m-maleimidobenzoyl-n-hydroxysuccinimide (MBS) ester and the sulfo-MBS derivative, succinimidyl 4-(N-maleimidomethyl)cyclohexane-1-carboxylate (SMCC), sulfo-succinimidyl 4-(N-maleimidomethyl)cyclohexane-1-carboxylate (sulfo-SMCC), carbodiimide, bisdiazonium-benzidine (BDB) or glutaraldehyde. The use of coupling agents is described in particular in the chapter “Production of Antisera Using Peptide Conjugates” of the reference book “The Protein Protocols Handbook” (2002) which is incorporated herein by reference.

When GMBS, MBS, SMCC or sulfo-SMCC are used, they are preferably attached to a cysteine (C), which if not present in the peptide sequence, can be added, in particular at its N-terminal or C-terminal end.

### Conjugate

Preferably, the polypeptide of the immunogenic conjugate according to the invention comprises, or consists of, the sequence SSKEALAENNLNLPK (SEQ ID NO: 17), more particularly the sequence ESSKEALAENNLNLPK (SEQ ID NO: 18), and still more particularly the sequence ESSKEALAENNLNLPKC (SEQ ID NO: 19), the sequence AESSKEALAENNLNLPKC (SEQ ID NO: 20) or the sequence CESSKEALAENNLNLPKC (SEQ ID NO: 21), to which is optionally added on the C-terminal side the additional peptide linker sequence EGEZ or Z defined above. Preferably also this polypeptide is linked to the above defined carrier protein CRM197 by a coupling agent defined above.

Thus, preferably, the immunogenic conjugate as defined above is of the following formula (I):

(I)
[(AESSKEALAENNLNLPKC)-GMB]-CRM197

in which:
the polypeptide consists of the sequence AESSKEALAENNLNLPKC.
the carrier protein is CRM197.
the polypeptide is covalently linked to the carrier protein via the N-γ-maleimidobutyryl- (GMB) coupling agent,
the brackets indicate that the polypeptide is cyclized by formation of a peptide bond between the C-terminal cysteine (C) and the N-terminal alanine (A),
the square brackets indicate that at least one polypeptide is bound to the carrier protein.

Also preferably, the immunogenic conjugate as defined above is of the following formula (II):

(I)
[Acetyl-(CESSKEALAENNLNLPKC)-(X)ᵢ-GMB]-CRM197,

in which:
the polypeptide consists of the sequence CESSKEALAENNLNLPKC-(X)ᵢ-Z,
the carrier protein is CRM197.
the polypeptide is covalently linked to the carrier protein via the N-γ-maleimidobutyryl- (GMB) coupling agent,
the brackets indicate that the CESSKEALAENNLNLPKC sequence portion of the polypeptide is cyclized by formation of a disulfide bond between the C-terminal and N-terminal cysteine (C),
Acetyl-indicates that the free —NH₂ function of the N-terminal cysteine (C) is protected by an acetyl group by forming an amide bond,
(X) represents any peptide sequence of 1 to 5 amino acids, in particular EGE,
i is 0 or 1,
Z represents a tyrosine (Y) or a cysteine (C).
the square brackets indicate that at least one polypeptide is bound to the carrier protein.

Also preferably, the immunogenic conjugate as defined above is of the following formula (III):

(III)
[Acetyl-(CESSKEALAENNLNLPKC)-(X)ᵢ-Y-GMB]-CRM197,

in which:
the polypeptide consists of the sequence CESSKEALAENNLNLPKC-(X)ᵢ-Y.
the carrier protein is CRM197,
the polypeptide is covalently linked to the carrier protein via the N-γ-maleimidobutyryl- (GMB) coupling agent,
the brackets indicate that the CESSKEALAENNLNLPKC sequence portion of the polypeptide is cyclized by formation of a disulfide bond between the C-terminal and N-terminal cysteine (C),
Acetyl-indicates that the free —NH₂ function of the N-terminal cysteine (C) is protected by an acetyl group by forming an amide bond,
(X) represents any peptide sequence of 1 to 5 amino acids, in particular EGE,
i is 0 or 1, in particular 0,
the square brackets indicate that at least one polypeptide is bound to the carrier protein.

Also preferably, the immunogenic conjugate as defined above is of the following formula (IV):

(IV)
Acetyl-(CESSKEALAENNLNLP KC)-(X)ᵢ-C-GMB]-CRM197,

in which:
the polypeptide consists of the sequence CESSKEALAENNLNLPKC-(X)ᵢ-C,
the carrier protein is CRM197,
the polypeptide is covalently linked to the carrier protein via the N-γ-maleimidobutyryl- (GMB) coupling agent.
the brackets indicate that the CESSKEALAENNLNLPKC sequence portion of the polypeptide is cyclized by formation of a disulfide bond between the C-terminal and N-terminal cysteine (C).
Acetyl-indicates that the free —NH₂ function of the N-terminal cysteine (C) is protected by an acetyl group by forming an amide bond,
(X) represents any peptide sequence of 1 to 5 amino acids, in particular EGE,
i is 0 or 1,
the square brackets indicate that at least one polypeptide is bound to the carrier protein.

### Pharmaceutical Composition, in Particular Vaccine Composition

Among the adjuvants which may be administered in conjunction with the immunogenic conjugate defined above or which may be present in the pharmaceutical composition, in particular vaccine composition, defined above, mention may be made of Alum (alumina hydroxide), MONTANIDE™ ISA 51 VG. MONTANIDE™ ISA 720 VG, any water-in-oil emulsion or any oil-in-water emulsion, as well as in general of all adjuvants known in the scientific literature.

It is also possible to administer immunomodulators, such as MP40 for example, together with the immunogenic conjugate according to the invention or to add immunomodulators, such as MP40 for example, to the pharmaceutical composition, in particular vaccine composition, according to the invention.

### Therapeutic Applications

The immunogenic conjugate according to the invention or the pharmaceutical composition, in particular the vaccine composition, comprising it, is intended for active immunization and can be administered to treat all diseases related to overproduction or overexpression of the inflammatory cytokine IL-6 by inducing the production of antibodies which will bind to the overproduced or overexpressed IL-6 to prevent it from acting.

Preferably, the diseases related to overproduction or overexpression of IL-6 according to the invention are selected from the group consisting of:
Chronic inflammatory bowel diseases such as Crohn's disease and hemorrhagic rectocolitis or ulcerative colitis;
Arthritic diseases such as rheumatoid arthritis, juvenile arthritis, psoriatic arthritis, osteoarthritis, refractory rheumatoid arthritis, chronic non-rheumatoid arthritis, ankylosing spondylitis;
Chronic inflammatory bone diseases or IL-6 related diseases, in particular osteoporosis or any bone resorption disease;
Chronic inflammatory or IL-6-related diseases associated with infection such as septic shock, endotoxin shock, sepsis, hepatitis C, malaria, AIDS or other HIV-related infections;
Chronic inflammatory or IL-6-related diseases of the cardiovascular system, such as atherosclerosis, reperfusion injury following ischemia, coronary artery diseases, vasculitis, such as Behcet's disease or Wegener's granulomatosis:
Autoimmune diseases such as scleroderma, systemic scleroderma, lupus erythematosus, in particular disseminated, multiple sclerosis, or psoriasis:
Transplant-related diseases, including graft-versus-host disease, graft rejection, and trauma;
Allergies, in particular allergic asthma, and skin disorders related to delayed hypersensitivity reactions;
Immunodeficiencies, such as idiopathic immunodeficiency (CVID);
Chronic inflammatory or IL-6 related diseases of the respiratory system, in particular respiratory distress syndrome or pulmonary fibrosis:
Cancers that have a chronic inflammatory or IL-6-related component, such as plamocytoma, colorectal cancer, ovarian cancer, lymphoproliferative syndromes, multiple myeloma, in particular refractory multiple myeloma, or myeloproliferative syndromes;
Diabetes, in particular juvenile diabetes;
Amyloidosis, in particular Alzheimer's disease;
Uveitis, in particular its recurrent form;
Cachexia; and
Endometriosis.

### Administration

Preferably, the amount of immunogenic conjugate according to the invention administered per administration, i.e. the unit dose of immunogenic conjugate administered, is from 3 ng to 3 g, more preferably from 300 ng to 900 μg, even more preferably from 15 μg to 450 μg and most preferably from 30 μg to 300 μg. These unit doses of conjugate correspond respectively to unit doses of polypeptide according to the invention preferably from 1 ng to 1 g, more preferably from 100 ng to 300 μg, even more preferably from 5 μg to 150 μg and most preferably from 10 μg to 100 μg.

Preferably, the pharmaceutical composition, in particular vaccine composition, according to the invention comprises the immunogenic conjugate according to the invention at a dose of 3 ng to 3 g, more preferably of 300 ng to 900 μg, even more preferably of 15 μg to 450 μg and most preferably of 30 μg to 300 μg. Preferably also the pharmaceutical composition, in particular vaccine composition, according to the invention comprises the polypeptide according to the invention at a dose of 1 ng to 1 g, more preferably of 100 ng to 300 μg, still more preferably of 5 μg to 150 μg and most preferably of 10 μg to 100 μg.

Advantageously, the doses according to the invention enable the production of antibodies directed against the polypeptide to be favored over antibodies directed against the carrier protein, i.e. to favor a ratio of the titer of antibodies directed against the polypeptide to the titer of antibodies directed against the carrier protein greater than 1. Without wishing to be bound to a particular theory, the inventors believe that if there are too many antibodies produced against the carrier protein, these antibodies block the immune response in subsequent immunizations performed with the immunogenic conjugate according to the invention, it is therefore advantageous to keep the titer of antibody response against the carrier protein sufficiently low relative to that of the antibodies induced against the polypeptide.

The administration of the immunogenic conjugate is possible by intravenous, intradermal, subcutaneous, intramuscular, mucosal, including intranasal, or intraperitoneal routes.

The dosing regimen may range from once every two weeks to once a year for priming, until a good anti-IL-6 or anti-IL-6R antibody response develops, and then with booster doses, for example, every two months to once a year apart to maintain satisfactory beneficial antibody activity.

Preferably, the individual according to the invention is an animal, in particular a mammal, in particular a human. Preferably, the individual according to the invention overproduces or overexpresses IL-6 or is at risk of overproducing or overexpressing IL-6. Preferably, the individual according to the invention is 50 years or older, 60 years or older, 70 years or older, 80 years or older, or 90 years or older.

The invention will be further explained with the following non-limiting examples.

### EXAMPLES

### Example 1

The chemically synthesized peptide (CESSKEALAENNLNLPKC)Y (SEQ ID NO: 22) is conjugated to the carrier protein CRM197 (Pfenex, USA) on the one hand and to the carrier protein KLH (Keyhole limpet hemocyanin, SIGMA) on the other.

The peptide corresponds to the 78-93 area of human IL-6 (underlined part above) with the addition of a cysteine and a tyrosine at the C-terminus. The cysteine allows cyclization (part in brackets) with the N-terminal cysteine of the peptide via a disulfide bond. Conjugation to the carrier proteins is performed via the tyrosine with bis-diazo-benzidine (BDB, PolyPeptide Laboratories, Strasbourg France) according to standard methods.

The production of these conjugates generally leads to a standard peptide/carrier protein mass ratio in the range of 1:2 for KLH and for CRM197 as determined by the Amino-Acid Analysis (AAA) assay method. In other words, it is determined that for every 300 micrograms of total mass of peptide conjugated to the carrier protein, there are 200 micrograms of KLH and 100 micrograms of peptide, and there are 200 micrograms of CRM197 and 100 micrograms of peptide for the conjugate to CRM197. These conjugates are tested in an immunization experiment in Swiss mice (Charles River Laboratories, Ecully, France) using MONTANIDE™ ISA 51 VG (or ISA 51 for short) (SEPPIC, France) as adjuvant, administering a mixture containing 50 microliters of MONTANIDE™ ISA 51 VG for 50 microliters of conjugate, intramuscularly, with an injected dose of 300 micrograms of conjugate for each immunization. Two groups of 8 mice are thus immunized at D0, D14, D28, D42 against the KLH-conjugated peptide and the CRM197-conjugated peptide. The mice are sacrificed at D54, their blood collected, and the sera thus prepared are evaluated for their human anti-IL-6 antibody levels measured by ELISA (Desallais et al. (2016) Sci. Rep. 6:19549). On the day of sacrifice, the mice are doing well (shiny coat, weight maintained, normal stool), showing that immunization against the immunogenic conjugates has no toxic effect at the dose used.

The anti-IL-6 antibody titers obtained for the 2 groups, KLH and CRM197, are summarized in the table below:

| | CRM197 | KLH |
|---|---|---|
| Average titers | 20000 | 4788 |
| Median titers | 10980 | 1608 |

It can be seen that for identical conjugate doses, anti-IL-6 antibody titers are significantly higher with the CRM197 conjugate compared to the KLH conjugate.

### Example 2

The same compounds as in Example 1 are tested in subcutaneous immunizations of macaque monkeys performed at D1. D15, D30. D45, with MONTANIDE™ ISA 51 VG as adjuvant, in a mixture of 250 microliters of MONTANIDE™ ISA 51 VG/250 microliters of conjugate, 300 micrograms of KLH conjugate and 300 micrograms of CRM197 conjugate are administered in each immunization to two groups of 4 animals, respectively.

Blood samples and serum preparation are taken at D57, and antibody titers to human IL-6 are measured by ELISA. Throughout the experiment, the monkeys were evaluated for their general condition and they are doing well (normal activity, satisfactory appetite, maintained weight, normal stool), which shows that immunization against the immunogenic conjugates has no toxic effect at the dose used.

The anti-IL-6 antibody titers obtained for the 2 groups, KLH and CRM197, are summarized in the table below:

| | CRM197 | KLH |
|---|---|---|
| Average titers | 20510 | 7498 |
| Median titers | 17514 | 12116 |

Again, we see that the titers obtained after immunization against CRM197 conjugate are significantly higher on average and median than those obtained for KLH.

### Example 3

The chemically synthesized peptide cyclo(LTKLQAQNQWLQDMC) (SEQ ID NO: 23) is conjugated to the carrier protein CRM197 or KLH via the —SH group of the cysteine. Conjugation is performed with the bi-functional coupling agent N-γ-maleimidobutyryl succinimide (GMBS, Thermo-Fisher 22309) according to standard methods (“User guide GMBS and SulfoGMBS” Thermo-Fisher). The peptide comprises the sequence 176-189 of human IL-6 (underlined portion above), to which a C-terminal cysteine is added, and is cyclized by lactamization (part in brackets above).

Production of these conjugates leads to an estimated standard peptide to carrier protein mass ratio of 1:2 for KLH and 1:2 for CRM197. In other words, it is determined that for every 300 micrograms of total peptide conjugate mass, there are 200 micrograms of KLH and 100 micrograms of peptide for the KLH conjugate, and similarly for the CRM197 conjugate (100 micrograms of peptide to 200 micrograms of CRM197). These conjugates are tested in an immunization experiment in mice using as adjuvant, MONTANIDE™ ISA 51 VG (50 microliters of MONTANIDE™ ISA 51 VG for 50 microliters of conjugate), by intramuscular route with a dose of 450 micrograms of conjugate for each immunization, 2 groups of 8 mice are immunized at D0. D14, D28. D42 against the KLH-conjugated peptide and the CRM197-conjugated peptide. The mice are sacrificed at D54, their blood collected, and the thus prepared sera are evaluated for their human anti-IL-6 antibody levels measured by ELISA. On the day of sacrifice, the mice are doing well (shiny coat, weight maintained, normal stool), showing that immunization against the immunogenic conjugates has no toxic effect at the dose used.

The anti-hIL-6 antibody titers obtained for the 2 groups, KLH and CRM197, are summarized in the table below:

| | CRM197 | KLH |
|---|---|---|
| Average titers | 12350 | 8480 |
| Median titers | 7608 | 4772 |

It can be seen that for identical conjugate doses, the antibody titers are once again significantly higher with the CRM197 conjugate than with the KLH conjugate.

### Example 4

The chemically synthesized peptide (CIDKQIRYIIDGISALRKETC)EGEC (SEQ ID NO: 24) is conjugated to the carrier protein CRM197 on the one hand or KLH on the other. The peptide comprises the sequence 53 to 71 of human IL-6 (underlined part above) flanked by two cysteines that allow the cyclization of the peptide by the formation of a disulfide bond (part in brackets above). Conjugation is achieved via the C-terminal cysteine using the bi-functional coupling agent N-γ-maleimidobutyryl succinimide (GMBS). A linker peptide of sequence EGEC is present.

The production of these conjugates leads to an estimated standard peptide to carrier protein mass ratio of 1:2 for KLH and 1:2 for CRM197. In other words, it is determined that for every 300 micrograms of total peptide conjugate mass, there are 200 micrograms of KLH and 100 micrograms of peptide for the KLH conjugate, and similarly for the CRM197 conjugate (100 micrograms of peptide to 200 micrograms of CRM197). These conjugates are tested during immunizations of two groups of 6 New Zealand White (NZW) rabbits (Charles River) performed at D1, D15, D45, with MONTANIDE™ ISA51 VG as adjuvant, subcutaneously. In this case rabbits are immunized with 450 micrograms of conjugate (peptide-KLH or alternatively peptide-CRM197) at each immunization. The volume injected with MONTANIDE™ ISA 51 VG is 400 microliters (200 microliters of MONTANIDE™ ISA 51 VG with 200 microliters of conjugate).

Blood samples and serum preparation from these samples are done at D60. Antibody titers to human IL-6 were measured by ELISA. On the day of sacrifice, the rabbits were doing well (shiny coat, weight maintained, normal stools), showing that immunization against the immunogenic conjugates had no toxic effect at the dose used.

The titles obtained for the 2 groups, KLH and CRM197, are summarized in the table below

| | CRM197 | KLH |
|---|---|---|
| Average titers | 11192 | 8756 |
| Median titers | 6328 | 5640 |

Again, we see that the titers obtained after immunization against CRM197 conjugate are significantly higher on average and median than those obtained for KLH.

### Example 5

The chemically synthesized peptides (A)

(SEQ ID NO: 25)
(CLQAQNQWLQDTMC)Y

### and (B)

(SEQ ID NO: 26)
(CLQAQNQWLQDTMC)EGEY

are conjugated to the CRM197 carrier protein. The peptide comprises the sequence spanning residues 179-189 of human IL-6 (underlined portion above), with cysteines added to the N- and C-terminus for cyclization. The peptide is cyclized by forming a disulfide bond between the cysteines (underlined portion), and coupling to CRM197 of these peptides is accomplished through the terminal tyrosine using The bi-functional coupling agent used is BDB. Peptide (B) is identical to peptide (A) except for a linker sequence consisting of the tripeptide of sequence EGE.

The production of these conjugates leads to an estimated standard peptide to carrier protein weight ratio of 1:2. In other words, it is determined that for every 300 micrograms of total conjugate weight, there are 200 micrograms of CRM197 and 100 micrograms of peptide.

These compounds are tested in the immunizations of two groups of 6 mice performed at D1, D15, D45, with MONTANIDE™ ISA 51 VG as adjuvant, subcutaneously. In this case the mice are immunized with 300 micrograms of conjugate for each immunization. The volume injected in MONTANIDE™ ISA 51 VG is 200 microliters (100 microliters of MONTANIDE™ ISA 51 VG with 100 microliters of conjugate).

Blood samples and serum preparation from these samples are made at D54. Anti-human IL-6 antibody titers are measured by ELISA. On the day of sacrifice, the mice are doing well (shiny coat, weight maintained, normal stool), showing that immunization against the immunogenic conjugates has no toxic effect at the dose used.

The titers obtained for the 2 groups are summarized in the table below:

| | With EGE | Without |
|---|---|---|
| | linker peptide | linker peptide |
| Average titers | 3675 | 2718 |
| Median titers | 1752 | 1146 |

It can be seen that the titers obtained after immunization against the peptide possessing the EGE linker peptide are significantly higher on average and median than those obtained for the peptide without the linker peptide.

### Example 6

The chemically synthesized peptides (A)

(SEQ ID NO: 27)
(CFQSGFNEETC)Y

### and (B)

(SEQ ID NO: 28)
(CFQSGFNEETC)EGEY

are conjugated to the CRM197 carrier protein. The peptides comprise the sequence 101 to 111 of human IL-6 (underlined part above, the cysteines (C) are native). The peptide is cyclized by forming a disulfide bond between the two cysteines. Coupling of these peptides to CRM197 is achieved through the terminal tyrosine using the bi-functional coupling agent BDB.

The production of these conjugates leads to an estimated standard peptide to carrier protein weight ratio of 1:2. In other words, it is determined that for every 300 micrograms of total conjugate weight, there are 200 micrograms of CRM197 and 100 micrograms of peptide.

These compounds are tested in immunizations of two groups of 6 mice performed at D1. D15. D45, with MONTANIDE™ ISA 51 VG as adjuvant, subcutaneously. In this case the mice are immunized with 300 micrograms of conjugate during each immunization. The volume injected with MONTANIDE™ ISA 51 VG is 200 microliters (100 microliters MONTANIDE™ ISA 51 VG with 100 microliters of conjugate).

Blood samples and serum preparation from these samples are made at D54. Anti-human IL-6 antibody titers were measured by ELISA. On the day of sacrifice, the mice are doing well (shiny coat, weight maintained, normal stool), showing that immunization against the immunogenic conjugates has no toxic effect at the dose used.

The titers obtained for the 2 groups are summarized in the table below:

| | With EGE | Without |
|---|---|---|
| | binding peptide | binding peptide |
| Average titers | 2648 | 980 |
| Median titers | 1083 | 368 |

Again, it can be seen that the titers obtained after immunization against the peptide possessing the EGE linker peptide are significantly higher on average and median than those obtained for the peptide without linker peptide.

### Example 7

The chemically synthesized peptide

(SEQ ID NO: 22)
(CESSKEALAENNLNLPKC)Y

is conjugated to the carrier protein CRM197 (Pfenex, USA) as already described in Example 1. It is tested at 3 doses in subcutaneous immunizations of 3 groups of 10 macaque monkeys performed at D0, D15, D30, D45, D60, and D75 with MONTANIDE™ ISA 51 VG as adjuvant, in a mixture of 250 microliters of MONTANIDE™ ISA 51 VG/250 microliters of conjugate: at 600 micrograms of conjugate (i.e. 200 μg of peptide), at 300 micrograms of conjugate (i.e. 100 μg of peptide), at 150 micrograms of conjugate (i.e. 50 μg of peptide).

Blood samples and serum preparation were made at D30. D45. D60. D75 and D90 and anti-human IL-6 and anti-CRM197 titers were measured by ELISA. In the following tables, the anti-IL6 and anti-CRM197 titers, as well as the ratios (anti-IL6/anti-CRM197) are presented for the 3 groups.

| | Group receiving 200 μg of conjugated peptide | | |
|---|---|---|---|
| | Anti-IL6 | anti-CRM | Ratio |
| D0 | — | — | — |
| D15 | — | — | — |
| D30 | 3921 | 7070 | 55% |
| D45 | 23214 | 20000 | 116% |
| D60 | 31358 | 44453 | 71% |
| D75 | 44143 | 81086 | 54% |
| D90 | 42907 | 88428 | 49% |
| Average | | | 69% |

| | Group receiving 100 μg of conjugated peptide | | |
|---|---|---|---|
| | Anti-IL6 | anti-CRM | ratio |
| D0 | — | — | — |
| D15 | — | — | — |
| D30 | 2452 | 3900 | 63% |
| D45 | 12000 | 6680 | 180% |
| D60 | 10185 | 9300 | 110% |
| D75 | 14192 | 13343 | 106% |
| D90 | 16841 | 14866 | 113% |
| Average | | | 114% |

| | Group receiving 50 μg of conjugated peptide | | |
|---|---|---|---|
| | Anti-L6 | anti-CRM | ratio |
| DO | — | — | — |
| D15 | — | — | — |
| D30 | 3320 | 3360 | 99% |
| D45 | 7445 | 3760 | 198% |
| D60 | 12000 | 12683 | 95% |
| D75 | 33625 | 11163 | 301% |
| D90 | 39115 | 22428 | 174% |
| Average | | | 173% |

Surprisingly, it can be seen that the group immunized with 150 μg of conjugate (i.e. 50 μg of peptide present in the conjugate), i.e. the lowest dose, shows anti-IL6 titers as good as those of the groups immunized with 600 μg or 300 μg of conjugate, and that in particular the ratios (anti-IL6/anti-CRM197) are better for this group at all time points than for the group immunized with 600 μg of conjugate (i.e. corresponding to 200 μg of peptide present in the conjugate).

### Example 8

The chemically synthesized peptide

(SEQ ID NO: 22)
(CESSKEALAENNLNLPKC)Y

is conjugated to the carrier protein CRM197 (Pfenex. USA) as already described in Example 1. It is tested at 5 different doses in subcutaneous immunizations of groups of immunized rabbits; 5 μg of peptide (i.e. 15 μg of conjugate), 7 μg of peptide (i.e. 21 μg of conjugate), 10 μg of peptide (i.e. 30 μg of conjugate), 15 μg of peptide (i.e. 45 μg of conjugate), 50 μg of peptide (i.e. 150 μg of conjugate). Rabbits were immunized at D0, D13, D43, and D73 by subcutaneous injection of a mixture of 250 microliters of MONTANIDE™ ISA 51 VG/250 microliters of conjugate.

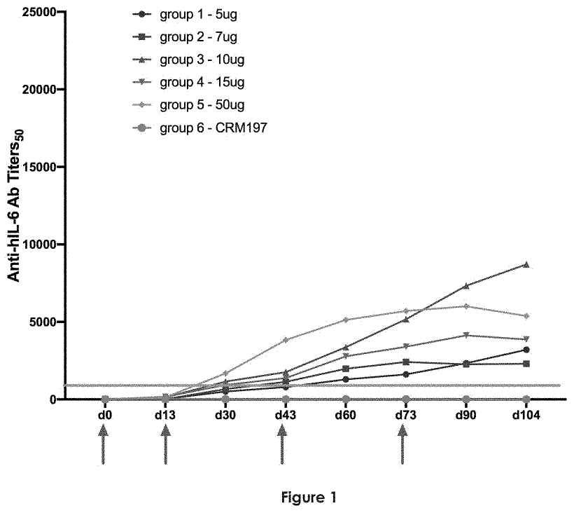

Sera were collected at D0, D13, D30, D43, D60, D73, D90, and D104 and antibody responses to IL6 measured by ELISA. The results of the immunizations are given in the graph of FIG. 1, in which each point represents the average of the titers obtained for the 5 rabbits of the group.

It can be seen that CRM197 alone does not induce anti-IL6 antibodies, but that all groups immunized with CRM197-conjugated peptide produce anti-IL6 antibodies. Surprisingly, we observe that the dose of 10 μg of peptide (i.e. 30 μg of conjugate) gives antibody levels equivalent to the dose of 50 μg of peptide (i.e. 150 μg of conjugate).

Summary of Sequence Identifiers:

| SEQ ID NO: | Description |
|---|---|
| 1 | Human IL-6 |
| 2 | Human IL-6 receptor alpha subunit |
| 3 | RYIIDGISALRKETCNKSNMC |
| 4 | NKSNMCESSKEALAENNLNLPK |
| 5 | AEKDGCFQSGFNEETC |
| 6 | FEVYLEYLQNRFESSEEQAR |
| 7 | NASLLTKLQAQNQWLQDM |
| 8 | RSFKEFLQSSLRALRQM |
| 9 | VLWEIVEMRKELCNGNSDCMN |
| 10 | NSDCMNNDDALAENNLKLPEIG |
| 11 | NDGCYQTGYNQEICLL |
| 12 | SYLEYMKNNLKDNKKDKARV |
| 13 | ALLTDKLESQKEWLRTKT |
| 14 | SLEEFLKVTLRSTRQT |
| 15 | EGEX |
| 16 | CRM197 |
| 17 | SSKEALAENNLNLPK |
| 18 | ESSKEALAENNLNLPK |
| 19 | ESSKEALAENNLNLPKC |
| 20 | AESSKEALAENNLNLPKC |
| 21 | CESSKEALAENNLNLPKC |
| 22 | CESSKEALAENNLNLPKCY |
| 23 | LTKLGAQNQWLODMC |
| 24 | CIDKQIRYIIDGISALRKETCEGEC |
| 25 | CLGAGNQWLGDMCY |
| 26 | CLQAQNQWLQDMCEGEY |
| 27 | CFQSGFNEETC |
| 28 | CFQSGFNEANDCEGEY |

1. An immunogenic conjugate comprising:
a carrier protein and
at least one polypeptide having at most 100 amino acids comprising a sequence of 5 to 50 amino acids of interleukin 6 (IL-6) or interleukin 6 receptor (IL-6R), or a variant sequence having at least 75% identity with the sequence of 5 to 50 amino acids of IL-6 or IL-6R,
wherein the polypeptide is covalently linked to the carrier protein and the carrier protein is a non-toxic mutant diphtheria toxin.

2. The immunogenic conjugate according to claim 1, wherein the carrier protein is CRM197.

3. The immunogenic conjugate according to claim 1, wherein the polypeptide comprises a 7 to 35 amino acid sequence of IL-6 or IL-6R or a variant sequence having at least 90% identity to the 7 to 35 amino acid sequence of IL-6 or IL-6R.

4. The immunogenic conjugate according to claim 1, wherein the polypeptide is cyclized.

5. The immunogenic conjugate according to claim 1, wherein the polypeptide comprises at least 5 amino acids of a sequence selected from the group consisting of sequences:
RYIIDGISALRKETCNKSNMC,
NKSNMCESSKEALAENNLNPK,
AEKDGCFQSGFNEETC,
FEVYLEYLQNRFESSEEQAR,
NASLLTKLQAQNQWLQDM,
and
SFKEFLQSSLRALRQM.

6. The immunogenic conjugate according to claim 1, wherein the polypeptide is linked to the carrier protein via a non-peptide coupling agent.

7. The immunogenic conjugate according to claim 1, of the following formula (I):
(I)
[(AESSKEALAENNLNLPKC)-GMB]-CRM197
in which:
the polypeptide consists of the sequence AESSKEALAENNLNLPKC,
the carrier protein is CRM197,
the polypeptide is covalently linked to the carrier protein via the N-γ-maleimidobutyryl- (GMB) coupling agent,
the brackets indicate that the polypeptide is cyclized by formation of a peptide bond between the C-terminal cysteine (C) and the N-terminal alanine (A),
the square brackets indicate that at least one polypeptide is linked to the carrier protein.

8. The immunogenic conjugate according to claim 1, of the following formula (II):
(II)
[Acetyl-(CESSKEALAENNLNLPKC)-(X)ᵢ-Z-GMB]-CRM197,
in which:
the polypeptide consists of the sequence CESSKEALAENNLNLPKC-(X)ᵢ-Z,
the carrier protein is CRM197,
the polypeptide is covalently linked to the carrier protein via the N-γ-maleimidobutyryl- (GMB) coupling agent,
the brackets indicate that the CESSKEALAENNLNLPKC sequence portion of the polypeptide is cyclized by formation of a disulfide bond between the C-terminal and N-terminal cysteine (C),
Acetyl-indicates that the free —NH₂ function of the N-terminal cysteine (C) is protected by an acetyl group by forming an amide bond,
(X) represents any peptide sequence of 1 to 5 amino acids,
i is 0 or 1,
Z represents a tyrosine (Y) or a cysteine (C),
the square brackets indicate that at least one polypeptide is bound to the carrier protein.

9. The immunogenic conjugate according to claim 1, for use in a method of therapeutic treatment or in a method of preventing or treating a disease related to overproduction of IL-6, in an individual.

10. The immunogenic conjugate according to claim 1, for use in a method of vaccination against IL-6 or IL-6R, or in a method of inducing an immune response against IL-6 or IL-6R, in an individual.

11. A pharmaceutical composition, in particular a vaccine composition, comprising as active substance at least one immunogenic conjugate as defined in claim 1, optionally in association with at least one pharmaceutically acceptable vehicle and/or excipient.

12. The pharmaceutical composition, in particular the vaccine composition, according to claim 11, further comprising at least one adjuvant.

13. The pharmaceutical composition, in particular the vaccine composition, according to claim 11, for use in a method of therapeutic treatment or a method of prevention or treatment of a disease related to overproduction of IL-6, in an individual.

14. The pharmaceutical composition, in particular the vaccine composition, according to claim 11, for use in a method of vaccination against IL-6 or IL-6R, or in a method of inducing an immune response against IL-6 or IL-6R, in an individual.

15. A method of preparing an immunogenic conjugate as defined in claim 1, comprising a step of covalently linking at least one polypeptide having at most 100 amino acids comprising a 5 to 50 amino acid sequence of interleukin 6 (IL-6) or IL-6 receptor (IL-6R), or a variant sequence having at least 75% identity to the 5 to 50 amino acid sequence of IL-6 or IL-6R with a carrier protein that is a non-toxic mutant diphtheria toxin.


SEQUENCE LISTING (28 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 3 - protein, 21 aa, Artificial sequence
  Squence d′IL-6
RYIIDGISALRKETCNKSNMC

SEQ ID NO: 4 - protein, 22 aa, Artificial sequence
  Squence d′IL-6
NKSNMCESSKEALAENNLNLPK

SEQ ID NO: 5 - protein, 16 aa, Artificial sequence
  Squence d′IL-6
AEKDGCFQSGFNEETC

SEQ ID NO: 6 - protein, 20 aa, Artificial sequence
  Squence d′IL-6
FEVYLEYLQNRFESSEEQAR

SEQ ID NO: 7 - protein, 18 aa, Artificial sequence
  Sequence d′IL-6
NASLLTKLQAQNQWLQDM

SEQ ID NO: 8 - protein, 17 aa, Artificial sequence
  Squence d′IL-6
RSFKEFLQSSLRALRQM

SEQ ID NO: 9 - protein, 21 aa, Artificial sequence
  Squence d′IL-6
VLWEIVEMRKELCNGNSDCMN

SEQ ID NO: 10 - protein, 22 aa, Artificial sequence
  Squence d′IL-6
NSDCMNNDDALAENNLKLPEIG

SEQ ID NO: 11 - protein, 16 aa, Artificial sequence
  Squence d′IL-6
NDGCYQTGYNQEICLL

SEQ ID NO: 12 - protein, 20 aa, Artificial sequence
  Squence d′IL-6
SYLEYMKNNLKDNKKDKARV

SEQ ID NO: 13 - protein, 18 aa, Artificial sequence
  Squence d′IL-6
ALLTDKLESQKEWLRTKT

SEQ ID NO: 14 - protein, 16 aa, Artificial sequence
  Squence d′IL-6
SLEEFLKVTLRSTRQT

SEQ ID NO: 15 - protein, 4 aa, Artificial sequence
  Lien peptidique
  MISC_FEATURE (4)..(4): Cys (C), Tyr (Y), Lys (K)
EGEX

SEQ ID NO: 16 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 17 - protein, 15 aa, Artificial sequence
  Variant de squence d′IL-6
SSKEALAENNLNLPK

SEQ ID NO: 18 - protein, 16 aa, Artificial sequence
  Variant de squence d′IL-6
ESSKEALAENNLNLPK

SEQ ID NO: 19 - protein, 17 aa, Artificial sequence
  Variant de squence d′IL-6
ESSKEALAENNLNLPKC

SEQ ID NO: 20 - protein, 18 aa, Artificial sequence
  Variant de squence d′IL-6
AESSKEALAENNLNLPKC

SEQ ID NO: 21 - protein, 18 aa, Artificial sequence
  Variant de squence d′IL-6
CESSKEALAENNLNLPKC

SEQ ID NO: 22 - protein, 19 aa, Artificial sequence
  Polypeptide selon l′invention
CESSKEALAENNLNLPKCY

SEQ ID NO: 23 - protein, 15 aa, Artificial sequence
  Polypeptide selon l′invention
LTKLQAQNQWLQDMC

SEQ ID NO: 24 - protein, 25 aa, Artificial sequence
  Polypeptide selon l′invention
CIDKQIRYIIDGISALRKETCEGEC

SEQ ID NO: 25 - protein, 14 aa, Artificial sequence
  Polypeptide selon l′invention
CLQAQNQWLQDMCY

SEQ ID NO: 26 - protein, 17 aa, Artificial sequence
  Polypeptide selon l′invention
CLQAQNQWLQDMCEGEY

SEQ ID NO: 27 - protein, 11 aa, Artificial sequence
  Polypeptide selon l′invention
CFQSGFNEETC

SEQ ID NO: 28 - protein, 15 aa, Artificial sequence
  Polypeptide selon l′invention
CFQSGFNEETCEGEY